(19) United States
(12) Patent Application Publication
(10) Pub. No.: US 2026/0315688 A1
(43) Pub. Date: Oct. 15, 2026

(54) Methods and systems for medicament volume verification

(71) Applicant: SHL Medical AG, Zug (CH)

(72) Inventors: Thomas Daniel James, Baltimore, MD (US); Christopher James Franzese, Boonton, NJ (US); Martin Michael Coyne, III, Boonton, NJ (US); Alejandra Linares Martinez, Boonton, NJ (US)

(21) Appl. No.: 19/483,636

(22) PCT Filed: May 24, 2024
(86) PCT No.: PCT/EP2024/064322

§ 371 Date: Nov. 12, 2025

Related U.S. Application Data
(60) Provisional application No. 63/471,039, filed on Jun. 5, 2023.

(30) Foreign Application Priority Data
Jul. 5, 2023 (EP) 23183610.7

(51) Int. Cl. A61M 5/142; A61M 5/36

(52) U.S. Cl. CPC A61M 5/142; A61M 5/365; A61M 2205/3331; A61M 2205/502

(57) ABSTRACT

The present disclosure provides a device configured to be removably coupled to a medicament container. The device includes a pump, a battery configured to supply power to the pump, at least one pressure sensor, at least one memory storage element, at least one processor, and data storage including program instructions stored thereon that when executed by the at least one processor, cause the drug delivery device to perform functions. The functions include (i) injecting, via the pump, a known quantity of fluid into a known volume surrounding the medicament container, (ii) measuring, via the pressure sensor, a pressure within the known volume surrounding the medicament container, (iii) determining, at least in part based on the measured pressure, a volume of a medicament within the medicament container, and (iv) providing for display, via a user interface, an indication of the volume of the medicament within the medicament container.

### TECHNICAL FIELD

The present disclosure generally relates to apparatus, systems, and methods for drug delivery devices, and more particularly to improved apparatus, systems, and methods to verify a volume of a medicament within a medicament container for use with a drug delivery device.

### BACKGROUND

Medications may be compounded—prepared in an aseptic manner—by pharmacy from vials or other bulk storage containers, such as vials. These medications are manipulated to accommodate fixed dosing, weight-based dosing, or body surface area dosing using syringes, needles, various adapters, and vial spikes.

Currently, a pharmacist or pharmacy technician prepares a given medicament compound (e.g., 50 mL of an oncology medication) and places it in a medicament container, such as a syringe, IV bag, or medication cassette. As each syringe of medication is added to the final container, the syringe is expelled and “pulled back” empty for later verification of the added volume. A second pharmacist then verifies the dosage, often by inspecting the syringes used. While gravimetric verification may be used, this is impractical given workflow and workload in busy compounding centers, in addition to the variable density of different medications.

Better solutions are needed to check for volumetric accuracy of filled medication containers and inspection for defects in the final medication container as part of current inspection workflows, while preserving the efficiency of staff involved in compounding operations. Additionally, as medications or medication containers are shipped through a supply chain before, during, or after dispensing, improved solutions are needed to examine medication containers for damage and suitability for use.

Additionally, medications such as oncology regimens may be dispensed in sequences that should be administered in order as predefined by a physician. During dispensing of a medication regimen, it is ideal for the pharmacist to encode the sequence (or verify the sequence) during dispensing to ensure proper/safe dispensing to a patient. It is also ideal for the pharmacist to encode the total number of medications within a sequence, also for quality control and safety purposes. Improved solutions in this area would enable the drug delivery device to enforce the proper administration sequence by an end user, preventing mis-ordered administration or incomplete administration of one or more medications.

### SUMMARY

The present invention is directed to improved apparatus, systems, and methods to verify a volume of a medicament within a medicament container for use with a drug delivery device. More particularly, the present disclosure is directed towards a standalone device for empirical verification of a filled volume prior to dispensing a compounded/filled medicament container to a patient and/or after dispensing the compounded/filled medicament container to the patient.

As such, in a first aspect, the present disclosure provides a device configured to be removably coupled to a medicament container. The device includes including a pump, a battery configured to supply power to the pump, at least one pressure sensor, at least one memory storage element, at least one processor, and data storage including program instructions stored thereon that when executed by the at least one processor, cause the drug delivery device to perform functions. The functions include (i) injecting, via the pump, a known quantity of fluid into a known volume surrounding the medicament container, (ii) measuring, via the pressure sensor, a pressure within the known volume surrounding the medicament container, (iii) determining, at least in part based on the measured pressure, a volume of a medicament within the medicament container, and (iv) providing for display, via a user interface, an indication of the volume of the medicament within the medicament container.

In a second aspect, the present disclosure provides a method comprising (i) injecting, via a device removably coupled to the medicament container, a known quantity of fluid into a known volume surrounding the medicament container, (ii) measuring, via at least one pressure sensor of the device, a pressure within the known volume surrounding the medicament container, (iii) determining, at least in part based on the measured pressure, a volume of a medicament within the medicament container, and (iv) providing for display, via a user interface, an indication of the volume of the medicament within the medicament container.

In a third aspect, the present disclosure provides a method comprising (i) after injection of a medicament via a device, determining a volume of the medicament within a medicament container of the device, and (ii) programming, via a communication interface associated with the medicament container, an indication of one or more disposal parameters of the device based at least in part on the determined volume of the medicament within the medicament container of the device.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 illustrates a simplified block diagram of a device, according to an example embodiment.

FIG. 2A illustrates a front view of an example device and an example medicament container, according to an example embodiment.

FIG. 2B illustrates a side view of the device and the medicament container of FIG. 2A, according to an example embodiment.

FIG. 2C illustrates a perspective view of the device and the medicament container of FIG. 2A, according to an example embodiment.

FIG. 2D illustrates a perspective view of the device and the medicament container of FIG. 2A with the device removably coupled to the medicament container, according to an example embodiment.

FIG. 3A illustrates a front view of another example device and an example medicament container, according to an example embodiment.

FIG. 3B illustrates a perspective view of the device and the medicament container of FIG. 3A, according to an example embodiment.

FIG. 3C illustrates a perspective view of the device and the medicament container of FIG. 3A with the device removably coupled to the medicament container, according to an example embodiment.

FIG. 4 illustrates a perspective view of another example device and an example medicament container, according to an example embodiment.

FIG. 5 is a block diagram of a method, according to an example embodiment.

FIGS. 6A-6C shows a schematic drawing of a process to evaluate disposal of a device after use, according to an example embodiment.

FIG. 7 is a block diagram of another method, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

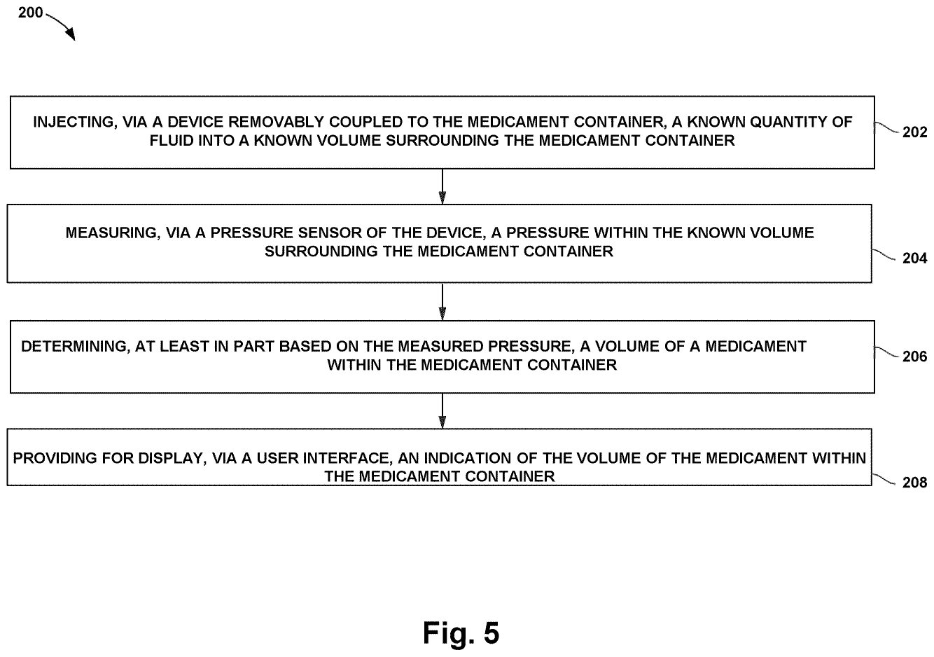

In FIGS. 5-7, referred to above, the blocks may represent operations and/or portions thereof and lines connecting the various blocks do not imply any particular order or dependency of the operations or portions thereof. It will be understood that not all dependencies among the various disclosed operations are necessarily represented. FIGS. 5-7 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

The present disclosure is directed towards a standalone device for determination of a filled volume prior to dispensing a compounded/filled medicament container to a patient and/or after dispensing the compounded/filled medicament container to the patient. The systems and methods described herein incorporate a volume verification device into pharmacy workflows to improve safety and efficiency of medicament volume verification. Such a volume verification device may be used to determine the filled volume (if any) without a priori knowledge of the filled volume or medication therein.

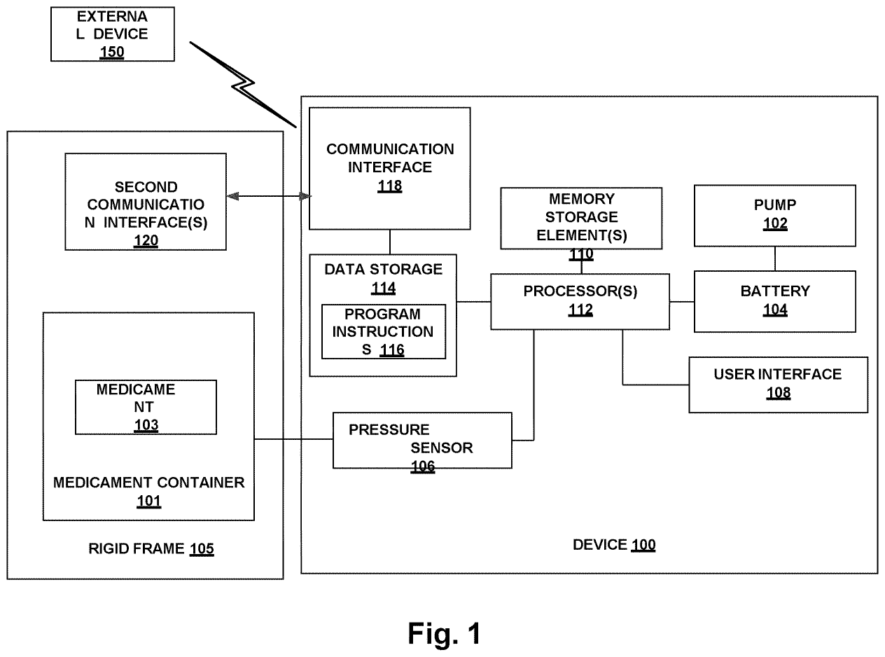

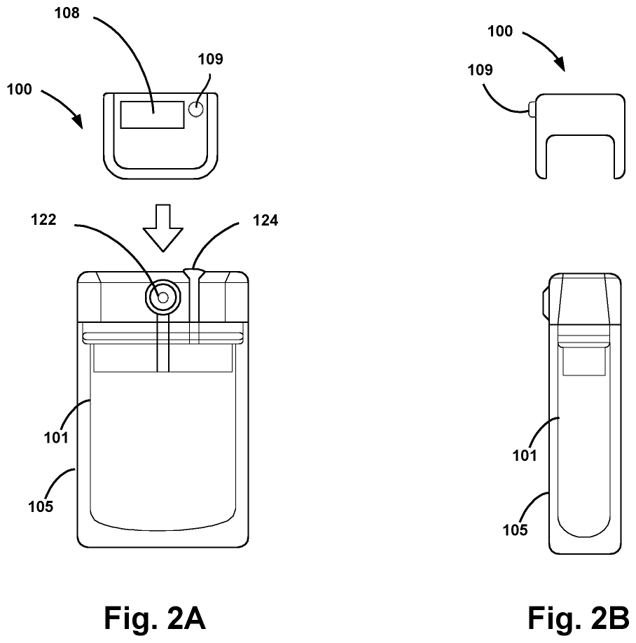

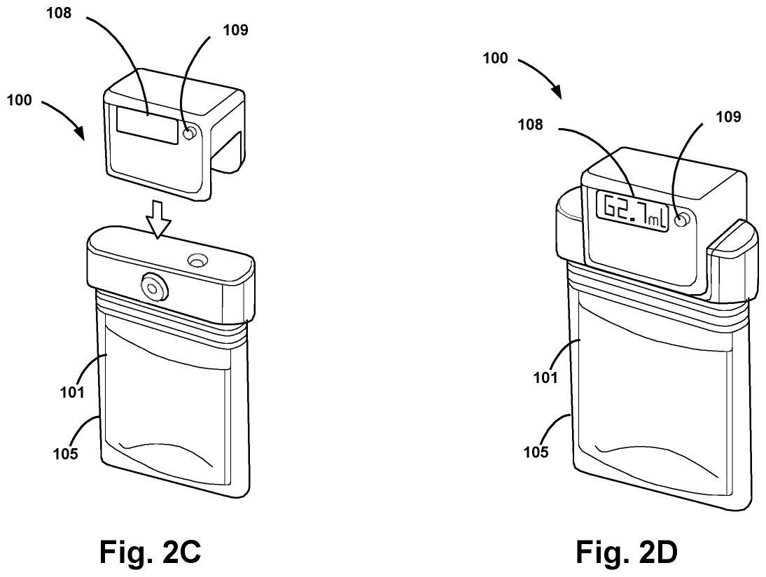

With reference to the figures, FIG. 1 illustrates a simplified block diagram of a device 100, according to an example embodiment. The device 100 is configured to be removably coupled to a medicament container 101, as discussed in additional detail below. In one example, the medicament container 101 comprises a flexible bag contained within a rigid frame 105 (e.g., a cassette). As shown in FIG. 1, the device 100 includes a pump 102 and a battery 104 configured to supply power to the pump 102. The pump 102 may comprise a piezoelectric air pump, as a non-limiting example. The device 100 further includes at least one pressure sensor 106 used to measure a pressure within a known volume surrounding the medicament container 101, as discussed in additional detail below. The device 100 also includes a user interface 108, such as an optical see-through display, an optical see-around display, a video see-through display, or a touch-screen display, as non-limiting examples. The user interface 108 may further include a button 109 or other user input means, as shown in FIGS. 2A-4. Alternatively, user interface 108 may be provided on a separate device, such as an electronic health record screen, companion application on a smartphone, or other computing device, as non-limiting examples.

The device 100 further includes at least one memory storage element 110. The at least one memory storage element 110 can include any type of memory now known or later developed including but not limited to volatile memory (such as RAM), non-volatile memory (such as ROM, flash memory, etc.) or any combination thereof. The device 100 also includes at least one processor 112 and data storage 114 including program instructions 116 stored thereon that when executed by the at least one processor 112, cause the device 100 to perform functions. Although various components of the device 100 are shown as distributed components, it should be understood that any of such components may be physically integrated and/or distributed according to the desired configuration of the system. Depending on the desired configuration, the at least one processor 112 can be any type of processor including, but not limited to, a microprocessor, a microcontroller, a digital signal processor, or any combination thereof.

In particular, the functions include (i) injecting, via the pump 102, a known quantity of fluid into a known volume surrounding the medicament container 101, (ii) measuring, via the at least one pressure sensor 106, a pressure within the known volume surrounding the medicament container 101, (iii) determining, at least in part based on the measured pressure, a volume of a medicament 103 within the medicament container 101, and (iv) providing for display, via the user interface 108, an indication of the volume of the medicament 103 within the medicament container 101. In one example, the fluid is a compressible fluid such as air or an ideal gas (e.g., hydrogen as a non-limiting example). In one example, the functions further include removing fluid from the volume surrounding the medicament container 101 to return the known volume surrounding the medicament container 101 to atmospheric pressure.

In one example, the volume of the medicament 103 within the medicament container 101 may be determined using the ideal gas law and its simplifications. Application of the ideal gas law is critical to volumetric system actions, (flow rate control, medication volume sensing), as volumes of abstract geometry cannot be directly probed by known cost-effective sensing methods. For a commercial presentation of this device, it is unlikely, however, that the system use pure ideal gases; use of ambient air is highly advantageous. So, while the ideal gas law, PV=nRT, applies directly to ideal gases, it is not a perfect representation of ambient air. Introducing a compressibility factor, z, to the Ideal Gas Law allows for general application to ambient air; PV=znRT. At the foreseeable system temperatures and pressures (280-310 K, 1-10 bar), 0.9992<z<1.0004, and therefore air can be approximated as an ideal gas.

To further simplify the equation, it is evident that the temperature term T, does not have great influence, since the foreseeable values are in a tight window of 280-310K. Even in the event that the volume of air starts at 280K and rises to 310K over the course of a volume-sensing action, the temperature term alone could not cause more than a +/−5% reduction in volumetric assessment accuracy. That is a very conservative case, as the control air temperature is likely to be dominated by the medicament container 101 enclosure temperature and indirectly by ambient room temperature (as the medicament container 101 has more internal surface area than the drug reservoir to conduct to the air). Furthermore, as the rigid frame, or “cassette” which contains the bag is vented to the ambient air throughout the preceding pharmacy workflow, the air within the cassette would be approximately equivalent to room temperature, which would not introduce any temperature-based inaccuracies. Allowing +/−5% measurement sensitivity in this step is likely acceptable clinically, and notably, a much greater sensitivity than volume verification methods in current practice, which contain no capability of empirical assessment. However, introduction of a system air temperature sensor, or a local cassette air temperature sensor would remove uncertainty due to a temperature difference between room-temperature air and the cassette's internal air. Obviously, although injections and infusions are used herein, accuracy of the system may be determined on a case by case basis, given a physiologic route of administration and appropriate clinical parameters thereto.

In a preferred system control model, the reduced ideal gas law becomes PV∝nR. Since nR represents the number of molecules in the system, or mass, m, the reduction is therefore PV∝m; V∝m/P. To determine void volume of the system at any given time, the system must keep track of the transferred mass to the control volume, e.g., via a known relationship between the drive parameters and the injection mass or via a comparative, proxy control region of known volume that air is dispensed from, such as a traditional accumulator model. In a volume assessment step, it is critical that the unfilled volume of the medicament cassette and the device's internal air volume is known. This is because the volume assessment device is assessing the system's air volume, and the filled volume of medicament is equal to the difference between an unfilled system's air volume and the filled system's air volume.

The ability to determine the volume of the medicament 103 within the medicament container 101 is particularly advantageous as the system is insensitive to initial fill, and can determined based only on the change in mass and without knowing the initial fill volume (i.e., no programming step is required). As such, the direct calculation of the volume of medicament 103 in the medicament container 101 allows the unique benefit of a blind, third-party verification of medication added to the medicament container 101, which is not a capability that exists in current pharmacy best practices for flexible drug reservoirs, e.g., bags for IV administration. In one application of this concept, the volume of medicament 103 in the medicament container 101 can be independently verified at-pharmacy with a dedicated device, without the introduction of human error, providing distinct benefit to pharmacy quality control practices.

In one example, the indication of the volume of the medicament 103 within the medicament container 101 comprises a numerical value (e.g., an indication of a number of mL). In another example, the indication of the volume of the medicament 103 within the medicament container 101 comprises providing an indication to a verifying pharmacist based on whether the filled volume is within the expected dose (±any tolerance, if desired). In other words, the pharmacist may, informed by data from the indicated volume and/or tolerance, choose to accept (i.e., dispense to a patient for later administration) or reject (i.e., do not dispense to a patient for later administration) the filled reservoir.

In one example, the device 100 further includes at least one first communication interface 118 configured to receive information from at least one second communication interface 120 positioned on a rigid frame of the medicament container 101 when the device 100 is removably coupled to the medicament container 101 via the rigid frame 105. In another example, the at least one second communication interface 120 is positioned directly on the medicament container 101. Such information may include an identification and/or characteristics of the medicament 103 in the medicament container 101. The at least one first communication interface 118 and the at least one second communication interface 120 may contain hardware to enable a communication link therebetween, such as processors, transmitters, receivers, antennas, etc., as discussed above. In one example, the communication link between the at least one first communication interface 118 and the at least one second communication interface 120 is a wired connection. In another example, the communication link between the at least one first communication interface 118 and the at least one second communication interface 120 is a wireless connection such as radio frequency identification (RFID), near-field communication (NFC), Bluetooth, Bluetooth low energy (BLE), Ultra wide band (UWB), wireless fidelity (Wi-Fi), cellular communication, and infrared (IR), as non-limiting examples.

In one example, the program instructions 116 further cause the device 100 to transmit, via the first communication interface 118, the volume of the medicament 103 within the medicament container 101 to an external device 150. The external device 150 may be any type of device that can receive data and display information corresponding to or associated with the data. For example, the external device 150 may be a mobile phone, a tablet, or a personal computer as examples. As such, the volume of the medicament 103 within the medicament container 101 may be transmitted to the prescribing facility, the dispensing facility, the drug manufacturer, the device manufacturer, and/or clinical trial administrator(s), as non-limiting examples.

In one example, the program instructions 116 further cause the device 100 to (i) receive an indication of an expected volume of the medicament 103 within the medicament container 101, and (ii) compare the determined volume of the medicament 103 within the medicament container 101 with the expected volume of the medicament 103 within the medicament container 101. In one example, the indication of the expected volume of the medicament 103 within the medicament container 101 is received via a user input at the user interface 108. In another example, the indication of the expected volume of the medicament 103 within the medicament container 101 is received via the second communication interface 120 on the rigid frame 105 at least partially surrounding the medicament container 101. In one specific example, the second communication interface 120 on the rigid frame 105 at least partially surrounding the medicament container 101 comprises a RFID/NFC chip (also referred to as a RFID/NFC tag). As such, the device 100 may read an embedded RFID/NFC chip on the rigid frame 105 to set the expected dose within the device 100 to verify against the filled volume within the medicament container 101. After measurement, the device 100 may be configured to program the embedded RFID/NFC chip on the rigid frame 105 with the measured volume of the medicament 103 in the medicament container 101. Other encoding methods than RFID/NFC may be used, as will be apparent to those skilled in the art.

In one example, the program instructions 116 further cause the device 100 to provide for display, via the user interface 108, an indication to proceed with a delivery of the medicament 103 within the medicament container 101 to a patient if the determined volume of the medicament 103 within the medicament container 101 is less than a threshold difference from the expected volume of the medicament 103 within the medicament container 101. The threshold difference between the determined volume of the medicament 103 within the medicament container 101 and the expected volume of the medicament 103 within the medicament container 101 may be ±0.5%, ±1%, ±1.5%, ±2%, ±3%, ±4%, ±5%, ±6%, ±7%, ±8%, ±9%, or ±10%, as non-limiting examples.

In another example, the program instructions 116 further cause the device 100 to provide for display, via the user interface 108, an indication to stop or prohibit a dispense of the medicament 103 within the medicament container 101 to a patient if the determined volume of the medicament 103 within the medicament container 101 is greater than or less than a threshold difference from the expected volume of the medicament 103 within the medicament container 101. The threshold difference between the determined volume of the medicament 103 within the medicament container 101 and the expected volume of the medicament 103 within the medicament container 101 may be ±0.5%±1%, ±1.5%, ±2%, ±3%, ±4%, ±5%, ±6%, ±7%, ±8%, ±9%, or ±10%, as non-limiting examples.

In one example, the program instructions 116 further cause the device 100 to detect, based at least in part on the measured pressure, an air leak rate of the medicament container 101. In one example, the program instructions 116 further cause the device 100 to compare the detected air leak rate to a threshold value or acceptable/unacceptable “decay” rate. The device 100 may then provide a “go/no-go” indication to a dispensing or verifying pharmacist based on whether the measured leak rate is acceptable. In other words, the pharmacist may, informed by data from the indicated leak rate, choose to accept (i.e., dispense to a patient for later administration) or reject (i.e., do not dispense to a patient for later administration) the filled reservoir.

In one example, the program instructions 116 further cause the device 100 to write, via its communication interface 118, parameters associated with the medicaments intended delivery to the medicament container's communication interface 120. These parameters may include the measured medicament volume, the prescribed medicament volume, the prescribed delivery profile (e.g. delivery flow rate or pressure), and/or information about any sequential or paired medication dispensing, such as the number of medications in the combinatorial delivery, the types of medications in said delivery, the medicament's delivery “position” within the combinatorial delivery, and/or instructions relating to the risk profiles of the medications in the combinatorial delivery. These parameters may be pulled from an external device or system, such as an EHR, or may be entered manually on the volume-assessment device. The encoding of these parameters onto the medicament container's communication interface 120 may allow for the downstream communication of these parameters to a drug delivery device with its own communication interface.

In one example, the device 100 described above is provided in a preferred embodiment with an air pump, pressure sensor, battery, controller, readout, and activation button. It may optionally include an electronic or mechanical “vent” valve. The device 100 also has an interface point to the air side (internal sealed volume) of the medicament container 101. The device 100 may optionally also have a numeric display, target volume setpoint selector, NFC or RFID reader (for communicating with an RFID/NFC chip associated with the medicament container 101), and/or interface electronics for communicating with an EHR system with a wired and/or wireless connection.

FIGS. 2A-2D show an illustrative version of the device 100 being assembled onto the medicament container 101. The device 100 device is placed over the top of the medicament container 101, providing a pneumatic path from the pump to the air connection 122, and blocking the fluid outlet 124. As a result, when the cassette is pressurized, fluid is not expelled from the cassette, as it would be with a pneumatically-driven delivery device; this affordance also protects sterility of the medicament 103 contained within the medicament container 101 as the volume is probed. Optionally, in an alternative workflow, the HCP may “cap” the flexible medication container, preventing drug leakage in the presence of a pressurized medicament container 101. The medicament container 101 may also be provided with a check valve, filter, or other means to prevent air ingress into the filled medicament container 101 due to fluid movement and resulting suction.

Optionally, in an alternative embodiment, the device 100 does not contain an air pump; rather the known “quantity of air” could be injected via an operator action (e.g., depressing a button that displaces a passive piston).

As discussed above, the device 100, once assembled onto the medicament container 101, may be used to verify or measure the volume of medicament 103 contained in the medicament container 101. This figure may be simply measured by displaying the filled volume, or may be verified if there is a target setpoint selected on the device 100. This verification may optionally confirm that the measured volume is within pre-defined or locally defined accuracy or tolerance bounds, representing both measurement uncertainty as well as medication dosing tolerance (for instance, ±5% of nominal, or another suitable figure, based on the dispensing facility or characteristics of the medication to be administered as discussed above). Alternatively, volume may be verified by measuring it using the probe, and comparing it to the expected volume (e.g., an expected volume that is contained on a RFID/NFC chip associated with the medicament container 101 as a non-limiting example). The expected volume and/or its tolerance bounds may be configured by the device manufacturer, the drug manufacturer, the prescribing facility, the dispensing facility, and/or any operators representing those entities.

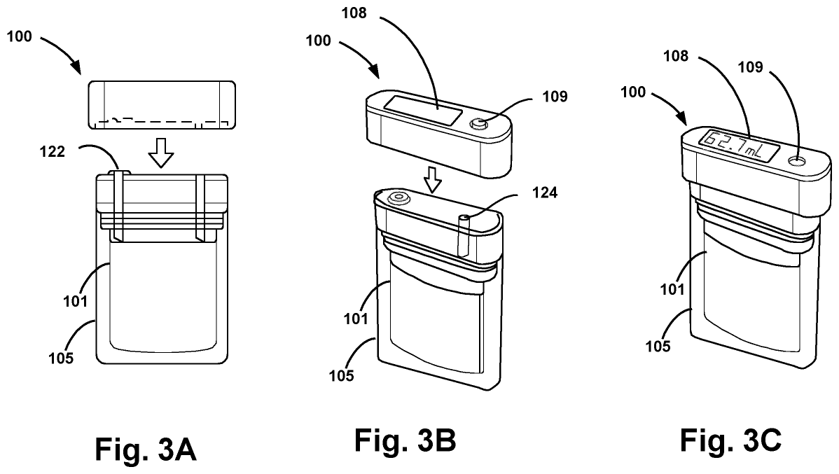

FIGS. 3A-3C are an alternative form embodiment of the device 100 of FIGS. 2A-2D. The device of FIG. 3A-3C is a top-reading view of the probed volume, which may be more ergonomic for workflow purposes, as multiple probed cassettes may be viewed side by side, which is particularly advantageous for verifying multiple medications in a regimen prior to dispensing.

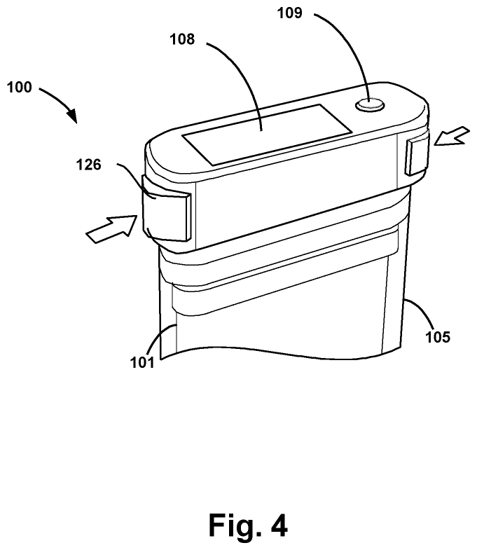

FIG. 4 shows an alternative removal method, whereby removal of the air pressure in the medicament container 101 happens passively when the release buttons 126 are pressed, which also releases the device 100 from the top connection with the medicament container 101.

As discussed above, some medications such as oncology regimens may be dispensed in sequences that should be administered in order as predefined by a physician. During dispensing of a medication regimen, it is ideal for the pharmacist to encode the sequence (or verify the sequence) during dispensing. It is also ideal for the pharmacist to encode the total number of medications within a sequence. Improved solutions in this area would enable the drug delivery device to enforce the proper administration sequence by an end user, preventing mis-ordered administration or incomplete administration of all medications.

In a first scenario, a set of cassettes is already provisioned with an ordinal sequence number. For instance, this may be done with stickers or clips containing a programmable RFID/NFC chip that are attached to each medicament container 101 during preparation by a pharmacist. Alternatively, this may be done by providing a programmable RFID/NFC chip in the medication cassette accessible to the apparatus described herein. In this scenario, the total number and order of medicament containers 101 is unknown a priori by the drug delivery system. Thus, the system needs to know a) that medicament container 101 are not duplicated (e.g., “1-2-3-4”, not “1-2-2-4” or “1-1-3-4”) and b) the total number of medicament container 101 in the sequence (1, 2, 3, . . . n).

In this scenario, during use of the device 100, the RFID/NFC chip is configured with the total number of cassettes, and the ordinal sequence of each cassette is verified as unique/unduplicated against the total expected number of cassettes in the sequence as the volume is probed. Further, the RFID/NFC chip is configured with the order of cassettes to detect a missing sequence (e.g., 1-3-4) where “2” is missing, or 1-2-4 where “3” is missing (and 4 should not be present).

In such an example, the program instructions 116 may further cause the device 100 to (i) set the total number of medications to be sequenced on the volume probe (e.g., via the selector switch or interface with an EHR system) corresponding to the total number of medications in the sequence as ordered by a physician, (ii) read the RFID/NFC chip associated with the medicament container 101 containing an ordinal sequence number, corresponding to the position of the medicament container 101 in the medication administration sequence as ordered by a physician, and verifying this number has not been duplicated (i.e., reused) during dispensing of the sequence, (iii) display an error to a user of the volume probe if a duplicated sequence number is detected during volume probe and/or RFID/NFC reading/writing, and/or (iv) in the case of a correct (i.e., valid) sequence position, program the RFID/NFC chip on the cassette with an integer corresponding to the total number of medications in the sequence as ordered by a physician.

In another example, the the program instructions 116 may further cause the device 100 to display, via the user interface, an error message if one or more of (i) a duplicated sequence number is detected, (ii) a missing sequence number is detected, or (iii) an unexpected sequence number is detected.

In a second scenario, a set of cassettes is provisioned with an RFID/NFC chip that is at least partially unprogrammed. The RFID/NFC chip may be partially programmed—for instance, with the drug name, rate, or volume—but is unprogrammed as to ordinal sequence number (i.e., position within a regimen/sequence) and/or total number of medications in the regimen/sequence.

In this second scenario, during use of the volume probe, the RFID/NFC chip is configured with the ordinal position of each cassette and total number of cassettes, both of which would be unknown a priori by the drug delivery system otherwise.

In such an example, the program instructions 116 may further cause the device 100 to (i) set the total number of medications to be sequenced on the device 100 (e.g., via the selector switch or interface with an EHR system) corresponding to the total number of medications in the sequence as ordered by a physician, (ii) program the RFID/NFC chip associated with the medicament container 101 with an ordinal sequence number, corresponding to the position of the medicament container 101 in the medication administration sequence as ordered by a physician, (iii) program the RFID/NFC chip associated with the medicament container 101 with an integer corresponding to the total number of medications in the sequence as ordered by a physician, and/or (iv) increment the sequence number after each volume probe and programming operation is successfully completed in preparation for the next volume probe and programming operation.

In another example, the program instructions 116 may further cause the device 100 to program the RFID/NFC chip associated with the medicament container 101 with one or more delivery instructions. These delivery instructions are then used by the device during delivery of the medicament. The one or more delivery instructions may include a flow rate setpoint, a pressure setpoint, an alarm volume, an indication of the types of errors to flag, an indication of the type of information to collect and transmit, sequence delay tolerances before an alarm, and a type of HER information to verify immediately ahead of dispense (such as in late-stage connectivity horizons), as non-limiting examples.

Various examples use cases for the device 100 described above will now be described in additional detail. The examples are representative in nature and are no way exhaustive or limiting.

### Cassette Leak Detection

While there are quality controls ensuring medicament container 101 robustness and critical-to-quality (CTQ) features in the manufacturing and assembling environments, there may be value in assessing that those CTQ features were not compromised in the interim secondary packaging, shipping, unboxing, or handling steps after the original medicament container 101 “checkout.” As the device 100 contains means of pressurization in addition to means of pressure sensing, an air leak rate could be determined by the device 100. The measured leak rate could be compared to reasonable bounds configured by the manufacturer or by appropriate standards (e.g., ISO, ASTM, USP, JP, or EP) to ensure the airtightness of the medicament container 101 has not been damaged or otherwise compromised before the dispensing of medication to the patient. In other words, the apparatus may be used for a variety of in-process quality control tests.

### Verification of Dispensed Dose for Clinical Trials

In dose-finding clinical trials, the burden of the variable dosing may be placed on the dispensing pharmacist or pharmacy technician. The device 100 could add an unbiased dataset of “true” dispensed dose to the clinical trial administrator, as the volume measurements would not be “polluted” by operator biases and/or rounding errors. This dataset could be immensely valuable to determining optimal dose ranges and sensitivities, with a level of accuracy never before accessible in pharmacy-fill clinical trial phases. In a similar application, fill volumes may be reported in a clinical trial (e.g., the “pivotal” or Phase III study) and once the drug goes to market to contribute to post-market surveillance activities.

### Calculation of Standard Fluid Volumes

In some instances, it may be preferable to flex medication concentration and instead fix the volume within the medicament container 101. In these instances, the device 100 may be used to identify the volume of medicament 103 in a medicament container 101, and then also be configured to calculate the volume to be added to “top up” (“QS” in pharmacy parlance) the fluid volume to a predetermined setpoint, usually with a diluent, as follows:
1. Set the device 100 to desired fixed volume (either by RFID/NFC chip reading with the cassette full volume, or by setting the volume probe manually)
2. Probe the volume of medicament 103 in the medicament container 101, as previously described
3. Calculate the difference between the max medicament container 101 volume and probed volume
4. Optionally, rounding the difference to correspond to the minimum dose scale on a syringe being used to add the QS diluent
5. Display the resulting amount to a user of the device 100
6. Optionally, re-probe the cassette to verify the correct QS amount has been added

TABLE 1

| | | Calculated | Optional |
|---|---|---|---|
| | | “QS” to Add | Display of QS |
| Volume Probe | Desired Final | to reach | volume to add |
| Result | Fixed Volume | fixed volume | (displayed on |
| (undiluted | (set by | (displayed on | volume probe, |
| medicament, | verifying HCP | volume probe, | based on method |
| method step 2) | method step 1) | method step 5) | step 4) |
| 37.5 mL | 50 mL | 12.5 mL | 13 mL* |
| 82.7 mL | 100 mL | 17.3 mL | 17 mL* |

*examples assume 20 mL syringe with 1 mL minimum scale increments

FIG. 5 is a block diagram of an example method 200, according to an example embodiment. Method 200 shown in FIG. 5 presents an embodiment of a method that could be used by the device 100 as described in FIGS. 1-4, as examples. Method 200 may include one or more operations, functions, or actions as illustrated by one or more of blocks 202-208. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 200 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 202, the method 200 includes injecting, via a device 100 removably coupled to the medicament container 101, a known quantity of fluid into a known volume surrounding the medicament container 101. As discussed above, in one example the fluid is a compressible fluid. At block 204, the method 200 includes measuring, via at least one pressure sensor 106 of the device 100, a pressure within the known volume surrounding the medicament container 101. At block 206, the method 200 includes determining, at least in part based on the measured pressure, a volume of a medicament 103 within the medicament container 101. At block 208, the method 200 includes providing for display, via a user interface 108, an indication of the volume of the medicament 103 within the medicament container 101.

In one example, the method 200 further includes removing fluid from the known volume surrounding the medicament container 101 to return the known volume surrounding the medicament container 101 to atmospheric pressure.

In another example, the method 200 further includes (i) receiving, via the user interface 108, an indication of an expected volume of the medicament 103 within the medicament container 101, and (ii) comparing the determined volume of the medicament 103 within the medicament container 101 with the expected volume of the medicament 103 within the medicament container 101. In one example, the indication of the expected volume of the medicament 103 within the medicament container 101 is received via a user input at the user interface 108. In another example, the indication of the expected volume of the medicament 103 within the medicament container 101 is received via a second communication interface 120 associated with the medicament container 101. In one example, the communication interface associated with the medicament container comprises a near-field communication (NFC) or radio frequency identification (RFID) tag.

In another example, the method 200 further includes providing for display, via the user interface, an indication to proceed with a delivery of the medicament 103 within the medicament container to a patient if the determined volume of the medicament 103 within the medicament container 101 is less than a threshold difference from the expected volume of the medicament 103 within the medicament container 101.

In another example, the method 200 further includes providing for display, via the user interface, an indication to stop a delivery of the medicament 103 within the medicament container 101 to a patient if the determined volume of the medicament 103 within the medicament container 101 is greater than a threshold difference from the expected volume of the medicament 103 within the medicament container 101.

In another example, the method 200 further includes preventing a dispense of the medicament 103 within the medicament container 101 to a patient if the determined volume of the medicament 103 within the medicament container 101 is greater than or less than a threshold difference from the expected volume of the medicament 103 within the medicament container 101. In one such example, preventing a dispense of the medicament 103 within the medicament container 101 to a patient comprises programming, via a communication interface associated with the medicament container 101, an indication that medicament container 101 is unsafe for use. Such an example provides a stopgap if a pharmacy were to accidentally dispense a medicament container 101 that “failed” testing with the probe.

In another example, the method 200 further includes enabling a delivery of the medicament 103 with the medicament container 101 to a patient if the determined volume of the medicament 103 within the medicament container 101 is less than a threshold difference from the expected volume of the medicament 103 within the medicament container 101.

In another example, the method 200 further includes programming, via the communication interface 120 associated with the medicament container 101, an indication that the determined volume of the medicament 103 within the medicament container 101 is less than a threshold difference from the expected volume of the medicament 103 within the medicament container 101.

In another example, the method 200 further includes providing for display, via the user interface 108, a difference between the determined volume of the medicament 103 within the medicament container 101 and the expected volume of the medicament 103 within the medicament container 101.

In another example, the method 200 further includes adding an amount of a diluent to the medicament container 101, wherein the amount of the diluent corresponds to the difference between the determined volume of the medicament 103 within the medicament container 101 and the expected volume of the medicament 103 within the medicament container 101.

In another example, the method 200 further includes (i) after injection of the medicament 103 via the device 100, injecting a second known quantity of fluid into the known volume surrounding the medicament container 101, (ii) measuring, via the at least one pressure sensor 106 of the device 100, a second pressure within the known volume surrounding the medicament container 101, (iii) determining, at least in part based on the measured pressure, a final volume of the medicament 103 within the medicament container 101, and (iv) comparing the determined volume of the medicament 103 within the medicament container 101 with the final volume of the medicament 103 within the medicament container 101.

In another example, the method 200 further includes providing for display, via the user interface 108, an indication of the final volume of the medicament 103 within the medicament container 101.

In another example, the method 200 further includes programming, via a communication interface 120 associated with the medicament container 101, a record that the medicament container 101 is not empty after injection of the medicament 103 via the device 100.

In another example, the method 200 further includes detecting, based at least in part on the measured pressure, an air leak rate of the medicament container 101. In another example, the method 200 further includes programming, via a communication interface 120 associated with the medicament container 101, an indication that the determined air leak rate is greater than a threshold difference from an acceptable air leak rate. In another example, the method 200 further includes preventing a dispense of the medicament 103 within the medicament container 101 to a patient if the determined air leak rate is greater than a threshold difference from an acceptable air leak rate.

### Medicament Container Disposal Example

The guidelines for safe management of healthcare waste are currently skewed towards landfilling and incineration as the safest options for most of the waste types generated. Some medicament containers 101, in normal use, are fully delivered to a patient, and thus are empty at the end of use. However, there may be instances where full delivery is not possible. For instance, medication delivery may be interrupted due to needle pullout, premature liftoff of the needle, or an adverse event, such as an infusion reaction.

Because the process of identifying which containers do and do not have medication is laborious, all used containers go into incineration to maximize safety. However, it would be useful to distinguish medicament containers that are completely empty from those that are partially full without a time-intensive or error prone process. Improved methods to distinguish these states could be used to shunt certain post-use containers to more sustainable recycling streams. For example, completely empty containers could be separated and reclaimed/recycled, while partially full containers could be properly incinerated.

As more advanced drug delivery devices become commonplace, there is information about the state of full drug delivery already present that can be advantageously used to improve this situation and divert empty containers into appropriate recycling or reclamation streams, reducing the total volume of waste incinerated.

Thus, in one example, the medicament container 101 may be programmed with medication status after administration, based on progress of drug administration with a drug delivery device. This information may be used (e.g., by scanning the used cassette) to distinguish full and empty cassettes after use and return. Users may then treat cassettes differently in terms of recycling/incineration based on the amount of medication they contain (e.g., shunting to recycling or reclamation in case of empty or partially full post-use reservoir, respectively).

In use, an airtight cassette of fixed volume is provided with an internally constrained flexible mediation container as previously described. The cassette (or other medicament container) is provided with an externally situated programmable RFID/NFC chip. The chip may be pre-configured (i.e., programmed) by pharmacy with certain information, such as the medication volume, flowrate, or sequence. The RFID/NFC chip is also re-programmable, as described further below.

A drug delivery system is provided with pneumatic drive interfacing with the sealed cassette. In addition, the drug delivery system contains RFID/NFC read/write capability, such as an RFID/NFC antenna and programmer. In other words, the drug delivery device can optionally read information from the cassette programmed during dispensing and write information onto the cassette during or after medication delivery using the apparatus.

Drug delivery may proceed in the following sequence:
1. Using the pneumatic drive, the cassette is probed at the beginning of the dispense. This starting value corresponds to the filled volume of the cassette (e.g., as filled by pharmacy prior to dispensing to the user).
2. Using the pneumatic drive, the medication is dispensed. As the dispense progresses, the volume is tracked (decrementing the full volume).
3. At the conclusion of dispense (either end of dose or error condition), the dispensed volume is compared to the full volume; alternatively, the volume in the cassette may be re-probed.
4. If the dispensed volume is approximately equal to the full volume (subject to some predetermined threshold), the cassette is empty, with only minimal (trace) residual volume. The drive controller re-programs the RFID/NFC chip on the cassette as empty.
5. If the dispensed volume is less than the full volume (subject to some predetermined threshold), the cassette contains meaningful residual medication. The drive controller re-programs the RFID/NFC chip on the cassette as partially full.
6. Steps 1-5 are repeated if multiple medications are administered in sequence; otherwise, the method proceeds.
7. The user sends all the cassettes to the takeback center; the takeback center scans all NFC chips upon receipt.
8. Cassettes that scan as “empty” can have materials separated and recycled.
9. Cassettes that scan as “partially full” are sent for incineration, for proper disposal of residual medicament, per local regulations.
10. Optionally, scan data (full/partial doses) may be uploaded to a patient electronic health record or patient support program for further intervention (e.g., patient coaching, adherence intervention, replacement dose for partial administration.

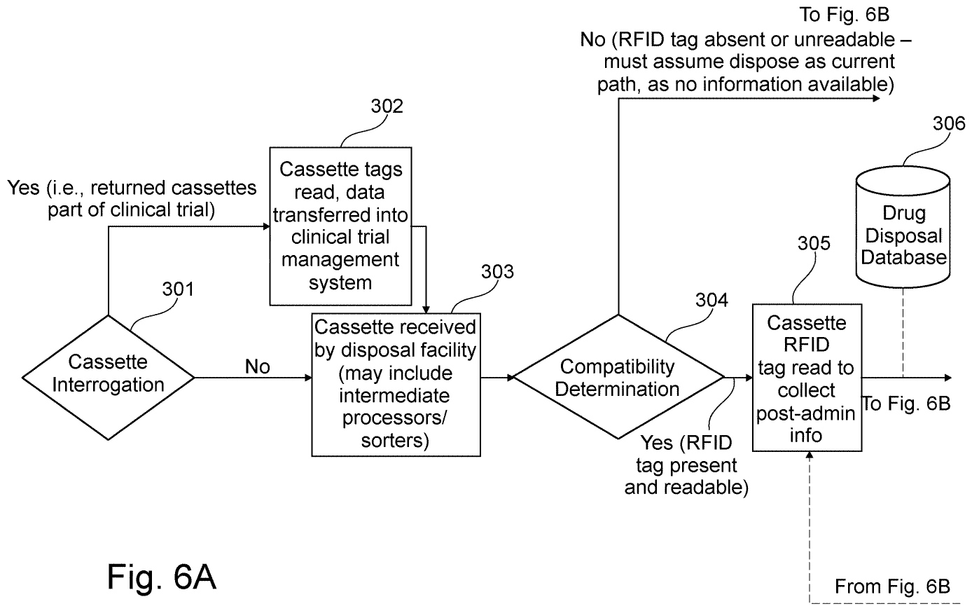

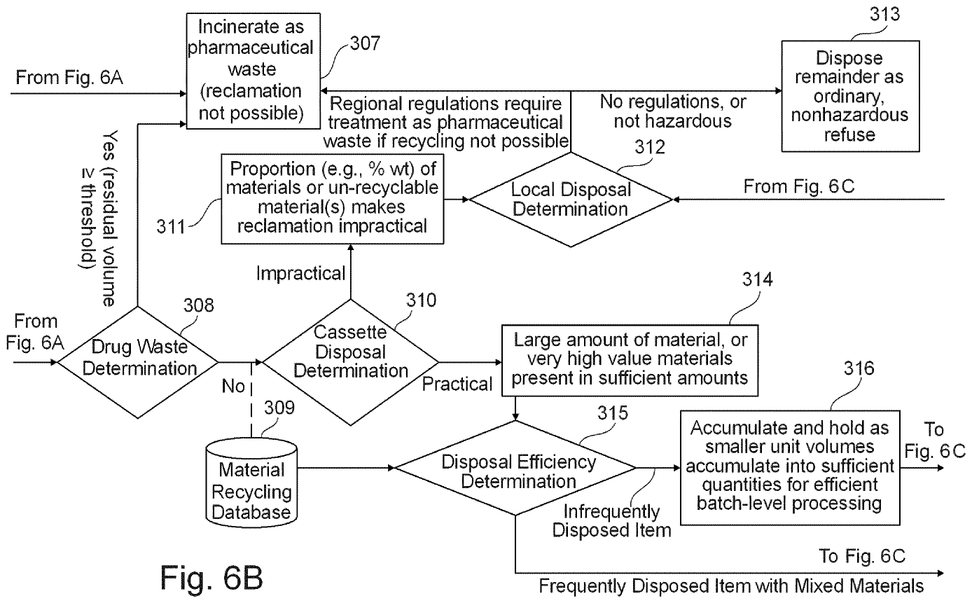

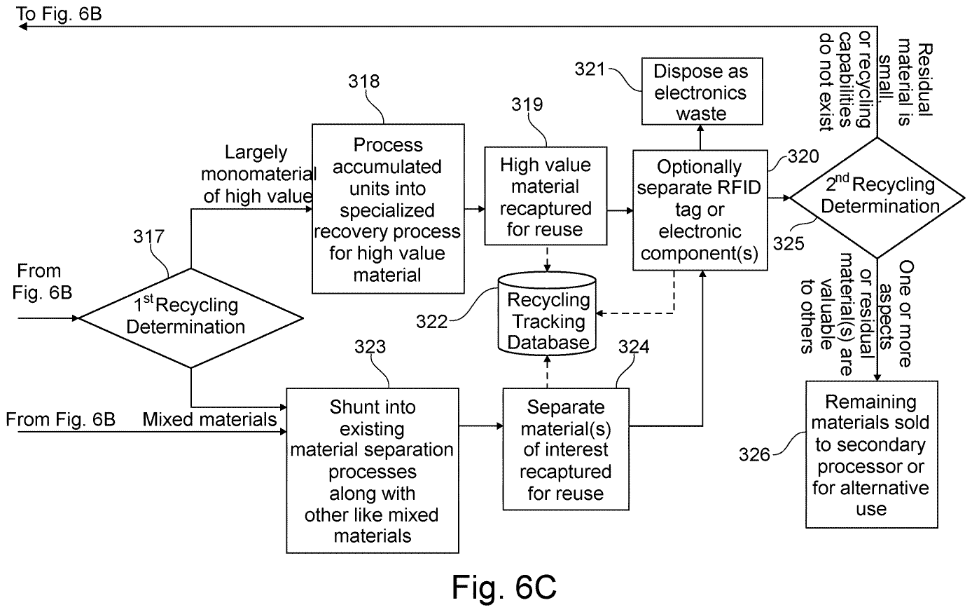

FIGS. 6A-6C shows a schematic drawing of a process to determine how to dispose of the device 100 after an injection is completed. As shown in FIG. 6A, the process may begin with block 301 with an interrogation of the cassette (e.g., device 100) to determine whether the cassette is part of a clinical trial. This step acts as a potential last ambush point to gather data post-administration from the cassette and to ensure all cassettes are returned (and potentially full doses administered). If it is determined that the cassette is part of a clinical trial, the process continues to block 302 with the cassette tags being read, and data is transferred into the clinical trial management system. The process then continues to block 303 where the cassette is received by a disposal facility (this may include intermediate processors and/or sorters). Block 303 could be done through a mail back process, by emptying out a sharps collector with multiple devices in it, or some other process. If it is determined that the cassette is not part of a clinical trial, the process moves directly to block 303.

The process continues at block 304 with a compatibility determination of the cassette to determine if the cassette includes the necessary disposal information. If a determination is made that there is an RFID tag (or other communication interface associated with the device) that is readable, the process continues at block 305 with reading the cassette RFID tag to collection post-administration information. In one example, the post-administration information includes one or more of a drug name or type, a volume of remaining medication, a container size, and regional information (such as a location of the device). The process may also include access to a drug disposal database 306. The drug disposal database 306 may include one or more threshold values (i.e., what is “empty”?) on general, drug-specific, regional level, or based on container size (e.g., % of max capacity remaining), as non-limiting examples. In one example, this information may be provided on the RFID tag at the point of manufacture. If a determination is made that there is not an RFID tag (or other communication interface associated with the device) or that the RFID tag is unreadable, the process continues in FIG. 6B at block 307 with incinerating the device as pharmaceutical waste as reclamation is not possible.

The process continues at block 308 with a drug waste determination, specifically, whether or not a threshold volume of medicament is still present in the medicament container of the device after administration of the injection. If the residual volume of the medicament in the medicament container of the device is greater than or equal to a threshold value, the process continues at block 307 with incinerating the device as pharmaceutical waste as reclamation is not possible. The process may also include access to a material recycling database 309. The material recycling database 309 may contain information on materials of construction of the device, resin type/subtype identifier, weight or proportion of one or more materials, manufacturer, presence/absence of electronic components or batteries, other custom recycling information (similar to 1-6 recycling codes in US). In one example, this information may be provided on the RFID tag at the point of manufacture.

If the residual volume of the medicament in the medicament container of the device is less than a threshold value, the process continues at block 310 with a cassette disposal determination. Specifically, at block 310 a determination is made whether or not it is practical to recycle the device based on one or more factors. If a determination is made that recycling of the device is impractical (e.g., at block 311 a proportion (e.g., % wt) of materials or un-recyclable material(s) makes reclamation impractical), the process continues to block 312 for a local disposal determination. The location disposal determination at block 312 takes into consideration the requirements of regional disposal regulations. If a determination is made that regional regulations require treatment of the device as pharmaceutical waste if recycling is not possible, then the process continues at block 307 with incinerating the device as pharmaceutical waste as reclamation is not possible. Conversely, if a determination is made that regional regulations allow treatment of the empty device as nonhazardous, the process continues at block 313 with disposing the remainder of the device as ordinary, nonhazardous refuse.

If, at block 310, a determination is made that recycling of the device is practical (e.g., at block 314 a determination is made that a large amount of material, or very high value materials are present in sufficient amounts in the device), the process continues to block 315 with a disposal efficiency determination. If a determination is made that the device is made up of an infrequently disposed item, the process continues at block 316 with accumulate and hold as smaller unit volumes accumulate into sufficient quantities for efficient batch-level processing. The process then continues in FIG. 6C with a first recycling determination at block 317. If a determination is made at block 317 that the device is largely monomaterial of high value, the process continues at block 318 with processing accumulated units into specialized recovery process for high value material. This captures the scenario where we have a cassette of largely single material (lots of one material). The process continues at block 319 with the high value material recaptured for reuse. This might focus on a single material of interest, or several. This branch is really about separating lots of a very expensive material. It may be possible that other materials could be left behind (e.g., stainless steel needles) or be shunted to secondary recovery (in this stream or by others). The process continues at block 320 with optionally separating the RFID tag or electronic component(s). The process continues at block 321 with disposing the RFID tag or electronic component(s) as electronics waste. The process may include a recycling tracking database 322, which may be used to track recycling activity over time. The recycling tracking database 322 may further be useful to support “net zero” claims or to support government credits/incentives.

If a determination is made at block 315 that the device is made up of frequently disposed item and/or with mixed materials, the process continues in FIG. 6C at block 323 with shunting into existing material separation processes along with other like mixed materials. If there is a lot of these materials consistently, this stream reduces down to continuous take-in-and-recycle, replacing the stockpile until a batch approach is appropriate. The shunting at block 323 could include a variety of separation techniques. For instance, it is possible to grind everything then separate with magnets for stainless steel components, by density or vibration for rubber/plastic/glass, etc. The separation techniques are exemplary only and are discussed for illustrative purposes only.

If a determination is made at block 317 that the device is made up of mixed materials, the process continues at block 323 with shunting into existing material separation processes along with other like mixed materials. Strategically, this is aimed at segregating waste (e.g., pens in one bin, cassettes in another, etc.) until a “batchable” mass is reached, which can then be reclaimed in one shot. This is one way of provisioning for a high unit volume in the future while still being cost effective to recycle at lower (initial) volumes. Put another way, efficiency may be preserved as high volume available is on a “continuous” basis. The process continues at block 324 with separating material(s) of interest to be recaptured for reuse. The process continues at block 320 with optionally separating the RFID tag or electronic component(s). The process continues at block 321 with disposing the RFID tag or electronic component(s) as electronics waste.

The process further continues from block 320 with a second recycling determination at block 325. If a determination is made at block 325 that any residual material is small or that any further recycling capabilities do not exist, the process continues to block 312 of FIG. 6B for a local disposal determination. If a determination is made that regional regulations require treatment of the device as pharmaceutical waste if recycling is not possible, then the process continues at block 307 with incinerating the device as pharmaceutical waste as reclamation is not possible. Conversely, if a determination is made that regional regulations allow treatment of the empty device as nonhazardous, the process continues at block 313 with disposing the remainder of the device as ordinary, nonhazardous refuse. If a determination is made at block 325 that one or more aspects of residual material(s) are valuable to others, the process continues at block 326 with the selling the remaining materials to a secondary processor or otherwise for alternative use.

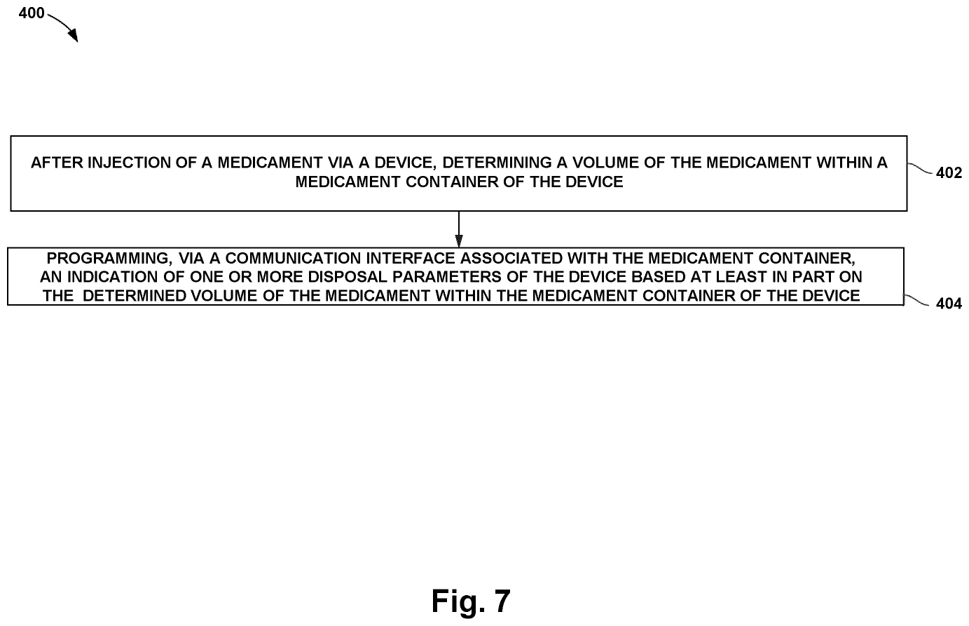

FIG. 7 is a block diagram of an example method 400, according to an example embodiment. Method 400 shown in FIG. 7 presents an embodiment of a method that could be used by the device 100 as described in FIGS. 1-4, as examples. Method 400 may include one or more operations, functions, or actions as illustrated by one or more of blocks 402-408. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 400 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 402, the method 400 includes, after injection of a medicament 103 via a device 100, determining a volume of the medicament 103 within a medicament container 101 of the device 100. At block 404, the method includes programming, via a communication interface 118 associated with the medicament container, an indication of one or more disposal parameters of the device 100 based at least in part on the determined volume of the medicament 103 within the medicament container 101 of the device 100.

In one example, the one or more disposal parameters comprise a record indicating whether (i) the medicament container 101 is empty or (ii) the medicament container 101 is not empty after injection of the medicament 103 via the device 100.

In another example, the one or more disposal parameters comprise a record indicating (i) the medicament container 101 should be recycled if a determination is made that the medicament container 101 is empty after injection of the medicament via the device 100 or (ii) the medicament container 101 should not be recycled if a determination is made that the medicament container 101 is not empty after injection of the medicament 103 via the device 100. In one example, the one or more disposal parameters comprise a record indicating the medicament container 101 should be incinerated or otherwise destroyed if a determination is made that the medicament container 101 is not empty after injection of the medicament 103 via the device 100.

In another example, the one or more disposal parameters comprise a record of a threshold residual volume associated with whether the medicament container 101 is empty or not empty after injection of the medicament 103 via the device 100.

In another example, the record of threshold residual volume is used as a comparator to determine whether the medicament container 101 is empty or not empty after injection of the medicament 103 via the device 100.

In another example, the one or more disposal parameters comprise an indication of one or more recycling and/or material processing steps based on a determination of one or more materials that make up the medicament container 101.

In another example, the method 400 further includes segregating, via the the communication interface, a medicament container 101 for one or more recycling or material processing steps.

In another example, the one or more disposal parameters comprise an indication of one or more materials that make up the medicament container 101, as discussed in additional detail above. In another example, the one or more disposal parameters comprise an indication of a location of the medicament container 101. Such information may be used to associate one or more local disposal requirements with the one or more disposal parameters to ensure that disposal of the medicament container 101 complies with local regulations.

In another example, the communication interface associated with the medicament container comprises a near-field communication (NFC) or radio frequency identification (RFID) tag, as discussed in additional detail above.

In another example, the method 400 further includes transmitting, via the communication interface 118, the indication of one or more disposal parameters of the device 100 to an external device.

It will be appreciated that other arrangements are possible as well, including some arrangements that involve more or fewer steps than those described above, or steps in a different order than those described above.

All embodiments described herein can be used for a device for measuring a medicament container containing medicament for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

Some other aspects of the invention are described by the following clauses.
1. A device configured to be removably coupled to a medicament container, comprising:
a pump;
a battery configured to supply power to the pump;
at least one pressure sensor;
at least one memory storage element;
at least one processor; and
data storage including program instructions stored thereon that when executed by the at least one processor, cause the device to:
   inject, via the pump, a known quantity of fluid into a known volume surrounding the medicament container;
   measure, via the pressure sensor, a pressure within the known volume surrounding the medicament container;
   determine, at least in part based on the measured pressure, a volume of a medicament within the medicament container; and
   provide for display, via a user interface, an indication of the volume of the medicament within the medicament container.
2. The device of clause 1, wherein the program instructions further cause the device to:
remove fluid from the known volume surrounding the medicament container to return the known volume surrounding the medicament container to atmospheric pressure.
3. The device of any one of clauses 1-2, wherein the indication of the volume of the medicament within the medicament container comprises a numerical value.
4. The device of any one of clauses 1-3, wherein the program instructions further cause the device to:
transmit, via a communication interface, the volume of the medicament within the medicament container to an external device.
5. The device of any one of clauses 1-4, wherein the program instructions further cause the device to:
receive an indication of an expected volume of the medicament within the medicament container; and
compare the determined volume of the medicament within the medicament container with the expected volume of the medicament within the medicament container.
6. The device of clause 5, wherein the indication of the expected volume of the medicament within the medicament container is received via a user input at the user interface.
7. The device of clause 5, wherein the indication of the expected volume of the medicament within the medicament container is received via a communication interface on a rigid frame at least partially surrounding the medicament container.
8. The device of clause 7, wherein the communication interface on the rigid frame comprises a near-field communication (NFC) or radio frequency identification (RFID) tag.
9 The device of any one of clauses 5-8, wherein the program instructions further cause the device to:
provide for display, via the user interface, an indication to proceed with a delivery of the medicament within the medicament container to a patient if the determined volume of the medicament within the medicament container is less than a threshold difference from the expected volume of the medicament within the medicament container.
10. The device of any one of clauses 5-8, wherein the program instructions further cause the device to:
provide for display, via the user interface, an indication to stop or prohibit a dispense of the medicament within the medicament container to a patient if the determined volume of the medicament within the medicament container is greater than or less than a threshold difference from the expected volume of the medicament within the medicament container.
11. The device of any one of clauses 1-10, wherein the program instructions further cause the device to:
detect, based at least in part on the measured pressure, an air leak rate of the medicament container.
12. The device of any one of clauses 1-11, wherein the program instructions further cause the device to:
determine, via a communication interface associated with the medicament container, an indication of an ordinal sequence number corresponding to a position of the medicament container in a medication administration sequence.
13. The device of clause 12, wherein the program instructions further cause the device to:
display, via the user interface, an error message if one or more of (i) a duplicated sequence number is detected, (ii) a missing sequence number is detected, or (iii) an unexpected sequence number is detected.
14. The device of any one of clauses 1-13, wherein the program instructions further cause the device to:
program, via a communication interface associated with the medicament container, one or more delivery instructions for the device.
15. A method comprising:
injecting, via a device removably coupled to the medicament container, a known quantity of fluid into a known volume surrounding the medicament container;
measuring, via at least one pressure sensor of the device, a pressure within the known volume surrounding the medicament container;
determining, at least in part based on the measured pressure, a volume of a medicament within the medicament container; and
providing for display, via a user interface, an indication of the volume of the medicament within the medicament container.
16. The method of clause 15, further comprising:
removing fluid from the known volume surrounding the medicament container to return the known volume surrounding the medicament container to atmospheric pressure.
17. The method of any one of clauses 15-16, further comprising:
receiving, via the user interface, an indication of an expected volume of the medicament within the medicament container; and
comparing the determined volume of the medicament within the medicament container with the expected volume of the medicament within the medicament container.
18. The method of clause 17, wherein the indication of the expected volume of the medicament within the medicament container is received via a user input at the user interface.
19. The method of clause 17, wherein the indication of the expected volume of the medicament within the medicament container is received via a communication interface associated with the medicament container.
20. The method of clause 19, wherein the communication interface associated with the medicament container comprises a near-field communication (NFC) or radio frequency identification (RFID) tag.
21. The method of any one of clauses 17-20, further comprising:
providing for display, via the user interface, an indication to proceed with a delivery of the medicament with the medicament container to a patient if the determined volume of the medicament within the medicament container is less than a threshold difference from the expected volume of the medicament within the medicament container.
22. The method of any one of clauses 17-21, further comprising:
providing for display, via the user interface, an indication to stop or prohibit a dispense of the medicament within the medicament container to a patient if the determined volume of the medicament within the medicament container is greater than or less than a threshold difference from the expected volume of the medicament within the medicament container.
23. The method of any one of clauses 17-22, further comprising:
programming, via the communication interface associated with the medicament container, an indication that the determined volume of the medicament within the medicament container is less than a threshold difference from the expected volume of the medicament within the medicament container.
24 The method of any one of clauses 17-23, further comprising:
providing for display, via the user interface, a difference between the determined volume of the medicament within the medicament container and the expected volume of the medicament within the medicament container.
25. The method of clause 24, further comprising:
adding an amount of a diluent to the medicament container, wherein the amount of the diluent corresponds to the difference between the determined volume of the medicament within the medicament container and the expected volume of the medicament within the medicament container.
26. The method of any one of clauses 16-25, further comprising:
after injection of the medicament via the device, injecting a second known quantity of fluid into the known volume surrounding the medicament container;
measuring, via the pressure sensor of the device, a second pressure within the known volume surrounding the medicament container;
determining, at least in part based on the measured pressure, a final volume of the medicament within the medicament container; and
comparing the determined volume of the medicament within the medicament container with the final volume of the medicament within the medicament container.
27. The method of clause 26, further comprising:
providing for display, via the user interface, an indication of the final volume of the medicament within the medicament container.
28. The method of any one of clauses 16-27, further comprising:
programming, via a communication interface associated with the medicament container, a record that the medicament container is not empty after injection of the medicament via the device.
29. The method of any one of clauses 16-28, further comprising:
detecting, based at least in part on the measured pressure, an air leak rate of the medicament container.
30. The method of clause 29, further comprising:
programming, via a communication interface associated with the medicament container, an indication that the determined air leak rate is greater than a threshold difference from an acceptable air leak rate.
31. The method of any one of clauses 29-30, further comprising:
preventing a dispense of the medicament within the medicament container to a patient if the determined air leak rate is greater than a threshold difference from an acceptable air leak rate.
32. The method of any one of clauses 16-31, further comprising:
preventing a dispense of the medicament within the medicament container to a patient if the determined volume of the medicament within the medicament container is greater than or less than a threshold difference from the expected volume of the medicament within the medicament container.
33. The method of clause 32, wherein preventing a dispense of the medicament comprises:
programming, via a communication interface associated with the medicament container, an indication that medicament container is unsafe for use.
34. The method of any one of clauses 16-33, further comprising:
enabling a delivery of the medicament with the medicament container to a patient if the determined volume of the medicament within the medicament container is less than a threshold difference from the expected volume of the medicament within the medicament container.
35. A method comprising:
after injection of a medicament via a device, determining a volume of the medicament within a medicament container of the device; and
programming, via a communication interface associated with the medicament container, an indication of one or more disposal parameters of the device based at least in part on the determined volume of the medicament within the medicament container of the device.
36. The method of clause 35, wherein the one or more disposal parameters comprise a record indicating whether (i) the medicament container is empty or (ii) the medicament container is not empty after injection of the medicament via the device.
37 The method of any one of clauses 35-36, wherein the one or more disposal parameters comprise a record indicating (i) the medicament container should be recycled if a determination is made that the medicament container is empty after injection of the medicament via the device or (ii) the medicament container should not be recycled if a determination is made that the medicament container is not empty after injection of the medicament via the device.
38. The method of any one of clauses 35-37, wherein the one or more disposal parameters comprise a record of a threshold residual volume associated with whether the medicament container is empty or not empty after injection of the medicament via the device.
39. The method of clause 38, wherein the record of threshold residual volume is used as a comparator to determine whether the medicament container is empty or not empty after injection of the medicament via the device.
40. The method of any one of clauses 35-39, wherein the one or more disposal parameters comprise an indication of one or more materials that make up the medicament container.
41. The method of any one of clauses 35-40, wherein the one or more disposal parameters comprise an indication of one or more recycling and/or material processing steps based on a determination of one or more materials that make up the medicament container.
42. The method of any one of clauses 34-36, wherein the communication interface associated with the medicament container comprises a near-field communication (NFC) or radio frequency identification (RFID) tag.
43. The method of any one of clauses 34-37, further comprising:
transmitting, via the communication interface, the indication of one or more disposal parameters of the medicament container to an external device.
44. The method of any one of clauses 34-37, further comprising:
segregating, via the the communication interface, a medicament container for one or more recycling or material processing steps.
45. The method of any one of clauses 34-38, wherein the one or more disposal parameters comprise an indication of a location of the medicament container.

1-14. (canceled)

15. A method comprising:
injecting, via a device removably coupled to the medicament container, a known quantity of fluid into a known volume surrounding the medicament container;
measuring, via at least one pressure sensor of the device, a pressure within the known volume surrounding the medicament container;
determining, at least in part based on the measured pressure, a volume of a medicament within the medicament container; and
providing for display, via a user interface, an indication of the volume of the medicament within the medicament container.

16. The method of claim 15, further comprising:
removing fluid from the known volume surrounding the medicament container to return the known volume surrounding the medicament container to atmospheric pressure.

17. The method of claim 15, further comprising:
receiving, via the user interface, an indication of an expected volume of the medicament within the medicament container; and
comparing the determined volume of the medicament within the medicament container with the expected volume of the medicament within the medicament container.

18. The method of claim 17, wherein the indication of the expected volume of the medicament within the medicament container is received via a user input at the user interface; or the indication of the expected volume of the medicament within the medicament container is received via a communication interface associated with the medicament container.

19. The method of any one of claim 18, further comprising:
programming, via the communication interface associated with the medicament container, an indication that the determined volume of the medicament within the medicament container is less than a threshold difference from the expected volume of the medicament within the medicament container.

20. The method of claim 18, further comprising:
providing for display, via the user interface, a difference between the determined volume of the medicament within the medicament container and the expected volume of the medicament within the medicament container.

21. The method of claim 15, further comprising:
after injection of the medicament via the device, injecting a second known quantity of fluid into the known volume surrounding the medicament container;
measuring, via the pressure sensor of the device, a second pressure within the known volume surrounding the medicament container;
determining, at least in part based on the measured pressure, a final volume of the medicament within the medicament container; and
comparing the determined volume of the medicament within the medicament container with the final volume of the medicament within the medicament container.

22. The method of claim 21, further comprising:
providing for display, via the user interface, an indication of the final volume of the medicament within the medicament container.

23. The method of claim 15, further comprising:
programming, via a communication interface associated with the medicament container, a record that the medicament container is not empty after injection of the medicament via the device.

24. The method of claim 15, further comprising:
detecting, based at least in part on the measured pressure, an air leak rate of the medicament container.

25. The method of claim 24, further comprising:
programming, via a communication interface associated with the medicament container, an indication that the determined air leak rate is greater than a threshold difference from an acceptable air leak rate.

26. The method of claim 24, further comprising:
preventing a dispense of the medicament within the medicament container to a patient if the determined air leak rate is greater than a threshold difference from an acceptable air leak rate.

27. The method of claim 15, further comprising:
preventing a dispense of the medicament within the medicament container to a patient if the determined volume of the medicament within the medicament container is greater than or less than a threshold difference from the expected volume of the medicament within the medicament container.

28. The method of claim 15, further comprising:
enabling a delivery of the medicament with the medicament container to a patient if the determined volume of the medicament within the medicament container is less than a threshold difference from the expected volume of the medicament within the medicament container.
